(19) United States
(12) Patent Application Publication
(10) Pub. No.: US 2021/0125194 A1
(43) Pub. Date: Apr. 29, 2021

(54) METHOD AND SYSTEM FOR COMPLETING CROSS-CHANNEL TRANSACTIONS

(71) Applicant: ALLCLEAR ID, INC., Austin, TX (US)

(72) Inventors: James Richard Holland, IV, Austin, TX (US); Andre Werup, Oslo (NO)

(73) Assignee: ALLCLEAR ID, INC., Austin, TX (US)

(21) Appl. No.: 17/079,188

(22) Filed: Oct. 23, 2020

Related U.S. Application Data
(60) Provisional application No. 62/925,089, filed on Oct. 23, 2019.

(51) Int. Cl. G06Q 30/00; H04L 9/30; H04L 9/32; G06Q 20/32; G06Q 20/38; G06Q 20/02; G06Q 20/40; G06K 7/10; H04L 29/08

(52) U.S. Cl. CPC G06Q 30/0185; G06Q 50/28; H04L 9/3242; H04L 9/3268; G06Q 20/326; G06Q 20/3829; G06Q 20/02; G06Q 20/40145; G06Q 20/3827; G06Q 20/3825; G06K 7/10366; H04L 67/26; H04L 2209/56; G06Q 2220/00; H04L 9/30

(57) ABSTRACT

A high security communication channel between the back-end application and the customer's mobile device is disclosed. An application programming interface that integrates into a service provider's back end application and a software development kit that integrates into a mobile application on the customer's mobile device establish a two-way communication channel between the back-end application and the mobile device. When a customer is ready to complete a transaction in one of the service provider's sales channels, such as online, by phone, in-person, by mobile device, or at a kiosk, the transaction moves to the mobile device for completion. A push message on the mobile device launches the service provider's mobile application and the customer completes the transaction quickly and securely using the advanced automation functions, such as biometrics, GPS, wallet, camera or near field communication, available on the mobile device.

This non-provisional application claims priority based upon prior U.S. Provisional Patent Application Ser. No. 62/925,089 filed Oct. 23, 2019 in the name of James Richard Holland, IV entitled “METHOD AND SYSTEM FOR COMPLETING CROSS-CHANNEL TRANSACTIONS,” the disclosures of which are incorporated herein in their entirety by reference as if fully set forth herein.

### BACKGROUND OF THE INVENTION

The present invention involves a novel improvement over the authentication technology described and claimed in U.S. Pat. No. 8,335,92, which includes one or more inventors in common with the present application. That patent describes an arrangement for utilizing a generally available personal data terminal as a secure and reliable authentication factor for user authentication. Also described is a method for secure transfer of data between two parties, a user and a service provider, where the user generates a unique authentication factor adapted for user authentication, called a user code, and the service provider registering the user's user code as an authentication factor. The described method is useful for various security services involving a user and a service provider in electronic channels where service providers are faced with the challenges of authenticating the users of their services.

However, beyond mere authentication, consumer-facing enterprises are under pressure to enable excellent customer experiences irrespective of where their customer engages—online, mobile, kiosk, in-person and teleservices. In response, savvy organizations are moving to a mobile-first experience using mobile technology in innovative ways to make the customer interaction with the enterprise more enjoyable while also ensuring better security.

Consumer authentication has traditionally been a clunky, insecure process that requires the end-user to either establish an account by entering the same set of personal information (name, address, email, phone number, payment card, etc.) or typing in their previously established username and password in order to sign in.

Over the years additional features have been added to the customer sign-in design with a focus on deterring fraud through improved security and identity assurance measures. Today, multi-factor authentication, identity proofing, behavioral analytics and the like are common elements on a robust customer identity experience.

But one feature of the design that has not seen significant improvements is the end-user experience. End-users repeatedly provide the same personal information with each service provider and continue to establish username/password credentials knowing how easy these are to exploit. And when credentials are forgotten, they must be reestablished through a convoluted process involving emails, texts, temporary passwords and other esoteric information. None of which does much to discourage a fraudster from overtaking an account.

Making the legacy customer identity workflow even less trustworthy is the fact that the processes for confirming a consumer's identity in the online and mobile environments are completely different than the processes for asserting one's identity over the phone, at the kiosk or in-person. Each customer touchpoint has its own customer identity assertion workflow, producing a mishmash of methods by which the customer authenticates themselves and introducing points of weakness that can be exploited.

While authenticating users is a necessary and useful part of any transaction, it is important that the overall transaction also be secure. There is a need, therefore, for a method and system that allows the authentication of a user while providing a secure environment in which to complete a transaction.

### SUMMARY OF THE INVENTION

In various embodiments, the present invention automates and secures the repetitive steps required to complete customer transactions, and not only provides secure, multi-factor identity authentication, but also supports digital signatures, authorizes payments and determines the location of the end user to coordinate logistics and improves the scheduling of delivery or pick-up.

In one embodiment, two components—an application programming interface that integrates into a service provider's back end applications and a software development kit that integrates into a mobile application on the customer's mobile device—establish a two-way, high security communication channel between the back-end application and the customer's mobile device. When a customer is ready to complete a transaction in one of the service provider's sales channels, the transaction moves to the customer's mobile device for completion. A push message launches the service provider's branded mobile application (or a co-branded application provided by a third party) and the customer completes the transaction in seconds.

More specifically, a customer may initiate a transaction in any sales channel, including online, by phone, in-person, by mobile device, or at a kiosk. The transaction information is processed through the provider's back end application and transmitted to a cross channel API. A high security, two-way communication channel connects the organization's application back-end application to the customer's mobile device. That connection allows that application to quickly and securely rely on the advanced automation functions found on the mobile device, such as biometrics, GPS, wallet, camera, near field communication, and Bluetooth, as well as the user's contacts, calendar and digital ID. Once the customer can be verified using one of these functions, the transaction may proceed.

Other embodiments of the present invention include a unique process for application attestation wherein intermediate push, optionally in connection with existing tools, such as SafetyNet, may be used to verify the security of a device.

One or more of the following steps may be incorporated into the intermediate push process: the application generates a random message key; the application encrypts the message key with the backend servers public key (provisioned in the application before it was published); the application sends its platform provided push address and encrypted message key to the backend server; the backend server uses the private key matching the public key the application was provisioned with to decrypt the message key (asymmetric encryption); the backend server generates a random session secret (nonce); the backend server calculates a message authentication code (MAC) using the message key as key and the session secret as message; stores the MAC on the session; the session secret is sent as the message content in a push request to the push service provider (e.g., Apple or Google) and this push send request also contains the application's unique push address and a package name or certificate tying this to a specific application as published trough the App Store (e.g., Google Play or Apple Appstore); the session secret is delivered by the push channel provider to the application (the push channel provider guarantees that only the application with a matching publisher/package name can receive the push message); the application calculates a hash message authentication code (HMAC) using the message key as key and the session secret as message; the application sends HMAC from step 9 to the backend server; and the backend server compares the client calculated HMAC with the server calculated HMAC. If they match the server knows that the client was the recipient of the session secret and that the recipient is the intended application as published in the App Store.

The foregoing has outlined rather broadly certain aspects of the present invention in order that the detailed description of the invention that follows may better be understood. Additional features and advantages of the invention will be described hereinafter which form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### DESCRIPTION OF THE DRAWINGS

For a more complete understanding of the present invention, and the advantages thereof, reference is now made to the following descriptions taken in conjunction with the accompanying drawings, in which:

FIG. 1 depicts a high-level view of one embodiment of the process for completing transactions of the present invention;

FIG. 2 depicts the process for approving a transaction using one embodiment of the present invention;

FIG. 3 depicts the process for approving a transaction using one embodiment of the present invention;

FIG. 4 shows a flow diagram of the process often encountered when entering a password to access a protected site or application;

FIG. 5 presents a summary of some of the functional features of various embodiments of the present invention;

FIG. 6 presents a summary of some of the security features of various embodiments of the present invention;

FIG. 7 diagrammatically presents several features commonly found on mobile devices which may be used in conjunction with various embodiments of the present invention;

FIG. 8 is a flow diagram showing the application attestation process flow of one embodiment of the present invention; and

FIG. 9 is a flow diagram showing the context binding process flow of one embodiment of the present invention.

### DESCRIPTION OF THE PREFERRED EMBODIMENTS

The present invention is directed to improved methods and systems for, among other things, transaction management. The configuration and use of the presently preferred embodiments are discussed in detail below. It should be appreciated, however, that the present invention provides many applicable inventive concepts that can be embodied in a wide variety of contexts other than the specific types of transaction management described herein. Accordingly, the specific embodiments discussed are merely illustrative of specific ways to make and use the invention, and do not limit the scope of the invention. In addition, the following terms shall have the associated meaning when used herein:

“application” is a software program designed to run on a mobile device;

“cloud” means a collection of logical devices which may or may not include underlying physical servers, wherein all such logical devices may be accessed without any knowledge, or with limited knowledge, of the underlying physical devices, and wherein the collection of logical devices has persistent logical resources, but is non-deterministic in its use of physical resources; and

“mobile device” means any portable computing device, typically having a display screen with touch input and/or a miniature keyboard.

Embodiments of the present invention, which are available as a cloud service or as an on-premise application, allow customers to initiate transactions in any sales channel and complete them quickly and securely using the advanced automation functions found on the customer's mobile device. A high security, two-way communication channel connects the organization's application back-end and the customer's mobile device and enables developers to create cross-channel transactions that start in one sales channel (e.g., website, call center, point-of-sale) and quickly finish on the customer's mobile device.

Various embodiments of the invention automate and secure the repetitive steps required to complete customer transactions, and not only provide secure, multi-factor identity authentication, but also support digital signatures, authorize payments and determine the location of the end user to coordinate logistics and improve the scheduling of delivery or pick-up. Customers can complete transactions quickly and securely without ever entering a username and password.

Replacing the username/password paradigm also ensures that a customer's time spent on consumer credential resets/recovery are significantly reduced while eliminating the need for the customer to recall usernames and passwords when instituted across all channels. The time and expense around this single activity is significant.

In one embodiment, the present invention consists of two components; an application programming interface, or API, that integrates into a service provider's back end applications and a software development kit, or SDK, that integrates into a mobile application on the customer's mobile device. These components establish a two-way, high security communication channel between the back-end application and the customer's mobile device. When a customer is ready to complete a transaction in one of the service provider's sales channels, the transaction moves to the customer's mobile device for completion. A push message launches the service provider's branded mobile application (or a co-branded application provided by a third party) and the customer completes the transaction in seconds.

The API provides high security access to a number of elements on the customer's mobile device, including, for example, the finger biometric touch pad, camera, and GPS functions. The SDK allows the authentication platform to be seamlessly integrated into the service provider's mobile application delivering uncompromising security and a passwordless user experience across all platforms, channels and devices—at Internet scale.

Because embodiments of the present invention employ the use of the customer's mobile device, those embodiments may be used in a wide variety of situations, such as:
Verifying customer location using GPS in support of logistics (finding nearest hospital, package delivery or pick-up, managing risk and or compliance);
Transmitting or updating personal contact information using contacts or related apps;
Transmitting payment information using a mobile wallet or RFID reader;
Collecting documents using a camera or RFID reader, such as for a driver's license, passport, utility bills, property deeds, titles and the like;
Scheduling events or appointments using a calendar app;
Exchanging health information using a health app and connected medical devices; and
Authorizing third parties and internet-of-things devices to access a resource using the smart home applications and other device or network management applications.

This invention has been shown to dramatically increase customer engagement while reducing transaction fraud.

As discussed above, embodiments of the present invention allow high value transactions that are initiated in one channel (at the service provider's website, call center or in-person/point-of-sale) to be completed or to “cross-over” to the mobile channel to leverage the advanced automation features on the customer's own mobile device. The customer uses the service provider's own mobile application, which is running the service described herein, to successfully authenticate their identity using the built in biometric read features on their device. Once the transaction is cleared the customer is brought back to their point of origination.

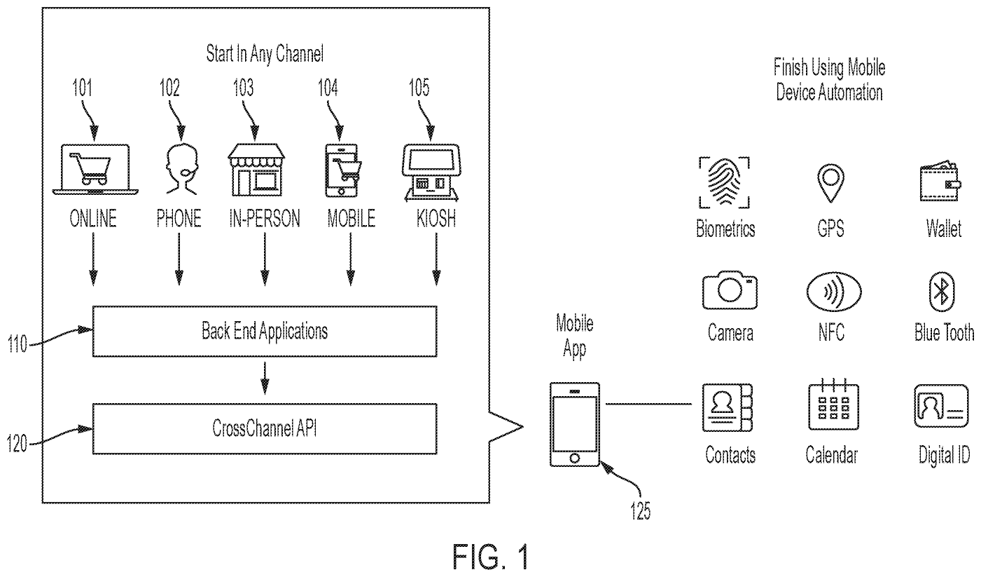

Referring now to FIG. 1 in which a customer may initiate transactions in any sales channel, including online 101, by phone 102, in-person 103, by mobile device 104, or at a kiosk 105. The information is processed through the provider's back end application 110 and transmitted to a cross channel API 120. A high security, two-way communication channel connects the organization's application back-end application 110 to the customer's mobile device 125. The connection to the mobile device 125 allows that application to quickly and securely rely on the advanced automation functions found on the mobile device, such as biometrics, GPS, wallet, camera, near field communication, and Bluetooth, as well as the user's contacts, calendar and digital ID. Once the customer can be verified using one of these functions, the transaction may proceed as indicated by the check mark on the mobile device 125.

### Example Application

In a retail customer check-out, the customer has completed their shopping experience on a retailer's website and is ready to check out. Instead of trying to remember their username and password and typing that information in or establishing a new account via the retailer's website, they are prompted at the checkout URL to identify themselves via the retailer's mobile application running the service of the present invention. A push notification is sent to the customer's smart phone, the customer clicks “OK” opening the retailer's mobile application and then provides their fingerprint or facial image for a biometric second factor. The application indicates “Success” and the website simultaneously indicates the authentication event has been successfully completed. The customer sees on the retailer's website, accessible through the URL, that all of the fields needed to complete the transaction have been auto-populated.

It is well known that username & password designs are easy to thwart, provide no assurance that the person logging in is the right person and are a consumer dissatisfier, not to mention costly. Biometric identity has begun replacing outdated, insecure and cumbersome username and password schemes in a multitude of industry sectors.

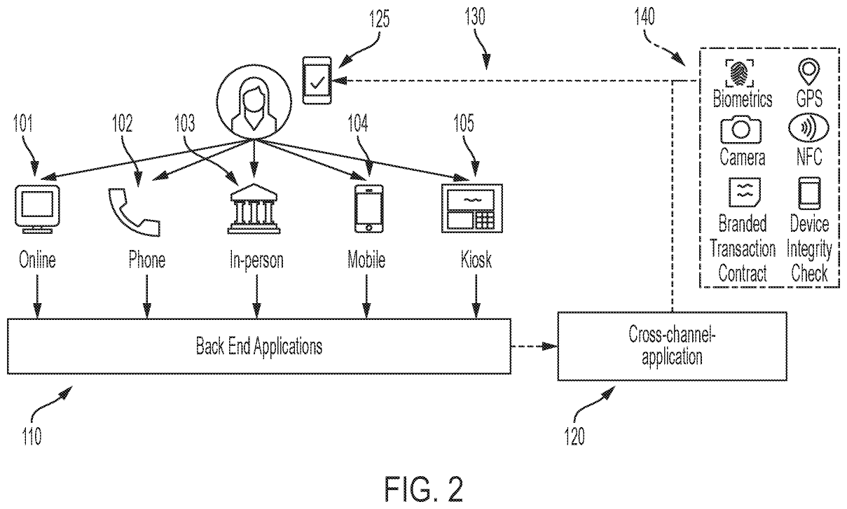

For example, and as shown in FIG. 2 in which a customer initiates a transaction in a sales channel, such as online 101, by phone 102, in-person 103, by mobile device 104, or at a kiosk 105. When the transaction is ready to be cleared, the back end application 110 pushes it through a cross channel API 120 to the customer's mobile device 125 for completion using the mobile device's advanced automation functions. The customer authorizes the transaction and transmits the necessary personal information through the mobile device 125. The connection to the mobile device 125 allows that application to quickly and securely rely on the advanced automation functions found on the mobile device 125, such as biometrics, GPS, wallet, camera, near field communication, and Bluetooth, as well as the user's contacts, calendar and digital ID. Once the customer can be verified using one of these functions, the transaction may proceed as indicated by the check mark on the mobile device 125.

In addition to the automation, cross-channel transactions are inherently more secure. To be successful, attackers must simultaneously compromise the front-end application, the cross-channel service and the strong multi-factor authentication functions on the customer's device. The result is a high trust, no password experience that increases customer engagement while radically reducing fraud and eliminating passwords.

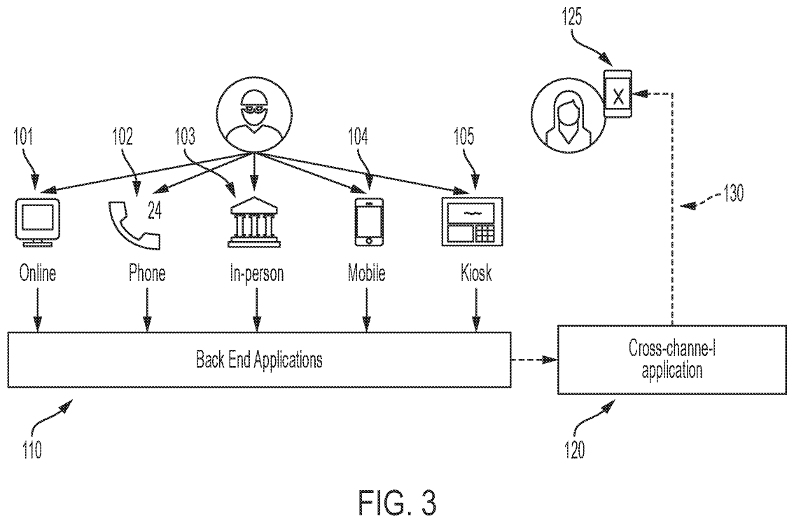

Referring now to FIG. 3 in which a customer initiates a similar transaction in a sales channel. In this case, a thief successfully attacks the organization's front-end application. The information is processed through the provider's back end application 110 and transmitted through a cross channel API 120 to the customer's mobile device 125 for authentication and verification. In this instance, the customer is unable to be verified or the customer is able to decline the transaction through the advanced automation functions on the mobile device 125. Because the customer cannot be verified or affirmatively declines the transaction, the transaction is declined as indicated by the “X” on the mobile device 125.

Industry sectors have their own unique challenges and requirements around their customer's identity. However, all want to achieve highly functional/intuitive consumer engagement, reduce instances and the costs associated with consumer identity fraud/data breaches, reduce customer support costs associated with onboarding, credentialing and re-establishing logins, adhere to best practices and/or address regulatory compliance, increase consumer utilization, and spot and address risk indicators associated with fraudulent identities in real-time.

### Financial/Banking Sector

Many people regularly apply strong, multi-factor authentication as they engage with their online and mobile banking applications. However, the process for kiosk and in-person interfaces are often different, as they use a physical ATM card and PIN for kiosk access and a combination of form factors for in-person transactions (ATM card, Driver's License, a physical check). Due to Anti-Money Laundering (AML) regulations, banks must have a high degree of confidence that their customers are who they say they are, plus they must know on an ongoing basis that the individual accessing services is eligible to do so. Embodiments of the present invention provide the financial institution with the ability to tightly bind the customer's proofed identity to their digital bank ID, preventing attempts at spoofing and ensuring proof of possession while also providing a singular, consistent method for customer authentication.

### Healthcare Sector

Even in countries with a national health ID, the process for patient identification is often not reliable and may be onerous for both the patient and the healthcare provider. Patients are asked to arrive to their doctor's appointment 15 minutes early to fill out registration forms, provide consents and authorizations and, most importantly, come prepared to provide some form of identity evidence such as their driver's license along with their health insurance card. Much of the workflow is manual and highly repetitive.

In the U.S., the patient identity landscape is changing with the advent of the Federal Trusted Exchange Framework and Common Agreement (TEFCA) regulations. In the not too distant future, healthcare organizations who wish to comport with the nationwide exchange framework will be required to assure patient identities to NIST Identity Assurance Level 2 (IAL2) and support Authentication Assurance Level 2 (AAL2).

Time spent registering at each clinic, hospital, lab and pharmacy will be a thing of the past as patients will be able to securely exchange their identity—already established and assured by one party—to other relying parties.

Mistaken identities and the creation of duplicate and overlaid medical records will also be controlled as the patient's digital ID ensures that only the right patient record is accessed. Inadvertent disclosures of protected health information to the wrong patient—a HIPAA violation—will also be avoided.

From appointment to treatment to payment, patients and their healthcare team will enjoy a more streamlined, safe and cost-effective method of transacting business as a result of utilizing the present invention.

### Retail

Consumer retail experiences involve repetitive, tedious steps to complete a transaction and may also involve multiple touchpoints such as ordering on-line, calling to check on status and then picking up at the store. Each data field that is required to be entered and each touchpoint the consumer has with the retailer is an opportunity for transaction abandonment. The more obstacles in the way of a fast and secure purchasing/check-out experience the more likely it is that the customer will simply give up. The present invention automates and secures the repetitive steps required to complete customer transactions.

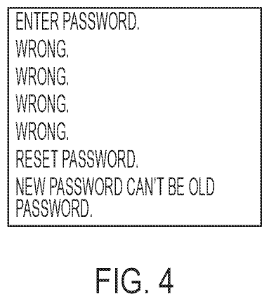

Nearly ⅓ of all online purchases are abandoned simply because the consumer can't recall their username and password in order to complete the transaction. The workflow in these instances is fairly typical. As shown in FIG. 4, the application requests that the user enter a password. When the user enters what they believe to be the correct password, it is flagged by the system as being wrong. The user attempts to re-enter the password a number of times and the system repeatedly flags the incorrect passwords as also being wrong. At some point, depending on the system's setting, the system will prompt the user to reset the password and remind the user that the new password cannot be the same as the old password, which is of little help when the user cannot recall the old password. Accordingly, Checkout usability in the form of a passwordless cross-channel workflow makes the online shopping experience easier for the consumer and reduces rates of abandonment.

Moreover, fewer form fields equal greater conversion. Conversion rate improves by almost half when the number of form fields to complete are reduced from 4 to 3. The present invention extracts oft-repeated data fields directly from the customer's own mobile device and wallet. Name, address, email, phone number, payment card details, etc. are all quickly pre-populated when using the present invention, leaving the customer with a more efficient and enjoyable one-click purchase experience.

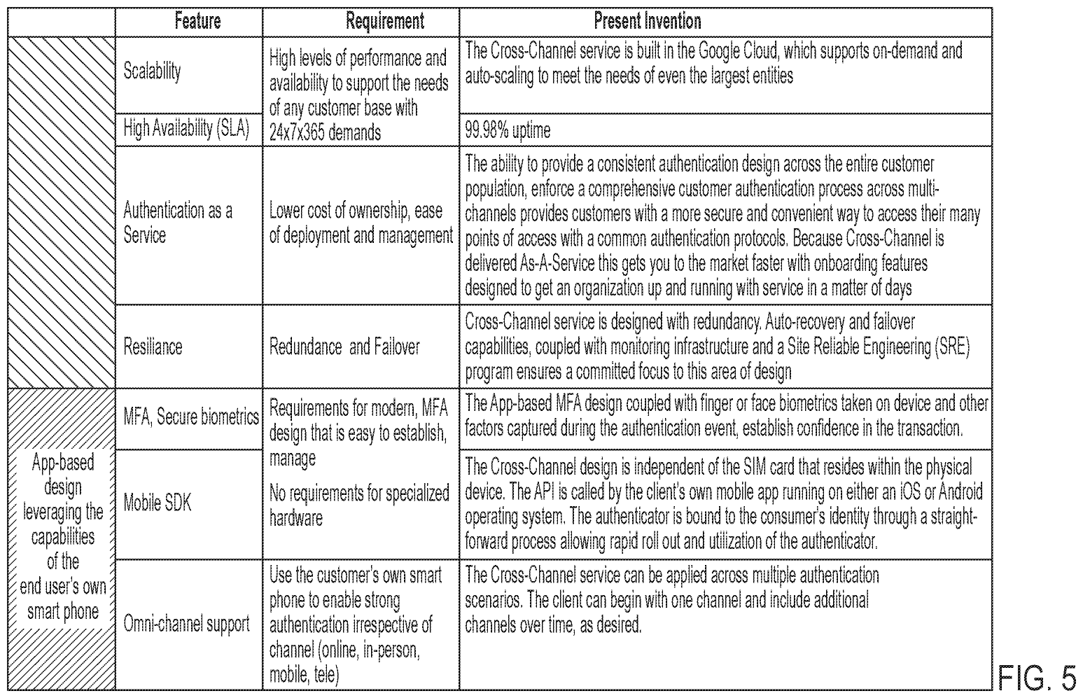

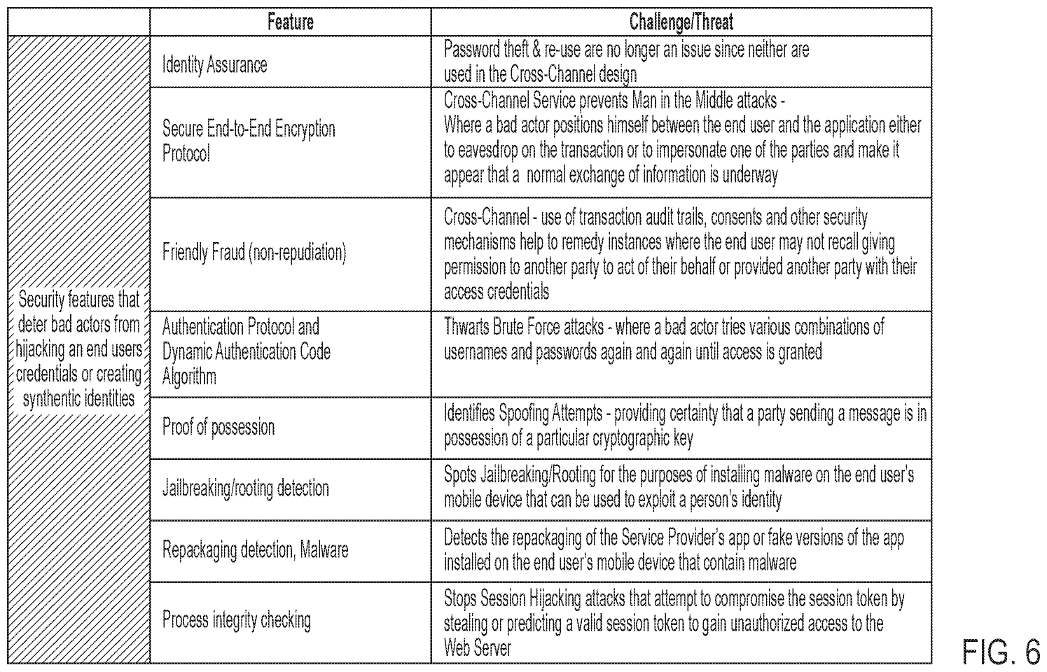

Various embodiments of the present invention provide numerous features and functionality. A summary of certain features, the underlying requirement for that feature, and the method utilized by embodiments of the present invention to provide the feature are described in FIG. 5. Similarly, there are a wide variety of security features provided by various embodiments of the present invention, and a summary of some of those features are summarized in FIG. 6.

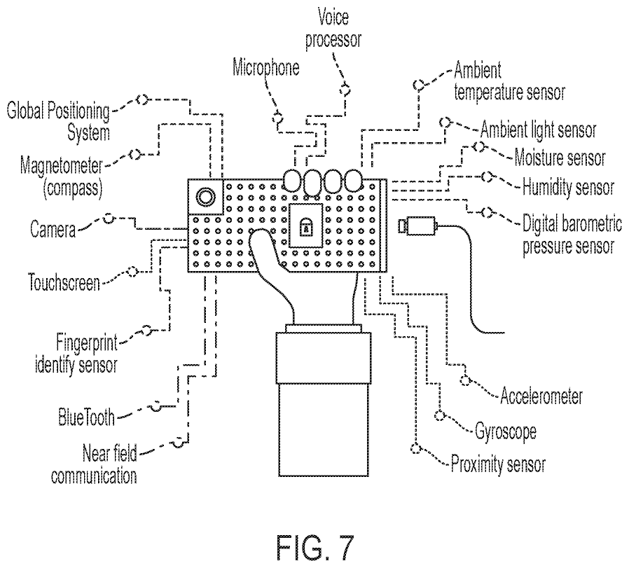

The rapid proliferation of mobile devices, the widespread availability of wireless network connectivity and the general use of cloud-based services has resulted in easy-to-provide, manage and use of mobile identity solutions. The set of sophisticated features found on most smart mobile devices today such as the camera, the fingerprint reader, GPS capabilities, NFC functionality and others allow the affiliated authentication service to be cognizant of a wide range of inputs that all assist in confirming the identity of the individual. A graphical depiction of some of the functionality found on many mobile devices today is shown in FIG. 7.

Embodiments of the present invention provide the ability to easily and securely identify customers to service providers using the mobile phone they already have in their possession.

### App Attestation

Most apps currently interact with remote services through APIs as their primary mode of operation. Although security concerns focus predominantly on user authentication, other means of attack must also be considered to ensure a secure API transaction.

Most operating systems provide native, device-level checks which can help prevent mobile application abuse. For example, the iOS operating system uses DeviceCheck to associate a few pieces of information per app with each device, and the Android operating system uses SafetyNet to determine if a device is running in a safe environment. These are useful capabilities, but they do little to create an in-depth mobile app and API protection strategy.

Fundamental to this strategy is that the API service must have confidence that the app running on the device is authentic and any unauthorized app must be rejected. Historically, the static API key was, and remains, the most common form of app authentication when making an API call. However, regardless of the methods used to protect the static API key, the API key is vulnerable through either reverse engineering in the app or by observing the key in an unprotected channel.

Remote mobile app attestation techniques can protect against both fake and tampered apps. Best practices dictate that the authenticity of an app should always be established outside the app itself in case the checks themselves are tampered with or bypassed. Accordingly, various embodiments of the present invention leverage two different mechanisms—intermediate push and SafetyNet—for application attestation. Both are optional and can be configured on or off by the service provider.

### Intermediate Push

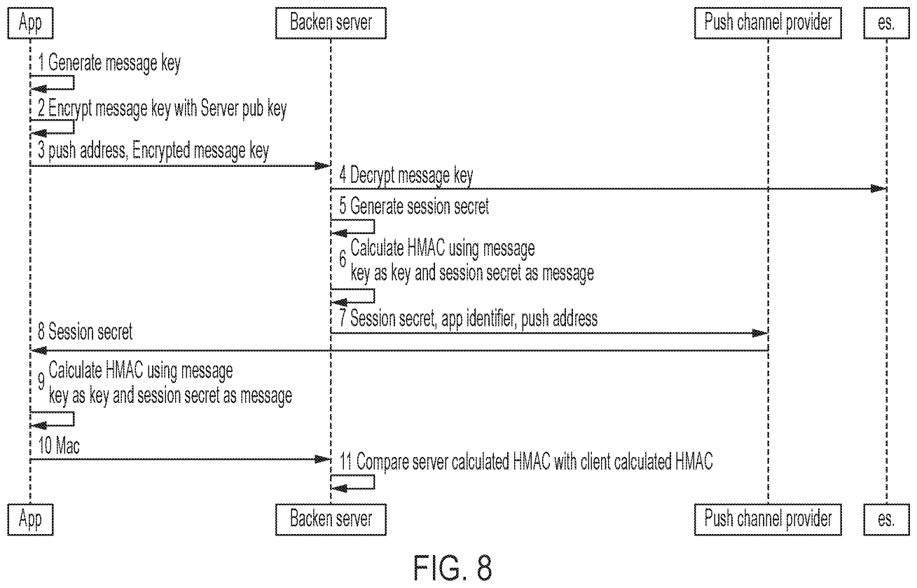

Leveraging the push channels provides delivery guarantee to ensure only authorized applications can make calls to the backend APIs. Referring now to FIG. 8, wherein one embodiment of the intermediate push process follows the following steps:

1. The application generates a random message key;

2. The application encrypts the message key with the backend servers public key (provisioned in the application before it was published);

3. The application sends its platform provided push address and encrypted message key from step 2 the to the backend server;

4. The backend server uses the private key matching the public key the application was provisioned with to decrypt the message key (asymmetric encryption);

5. The backend server generates a random session secret (nonce);

6. The backend server calculates a MAC using the message key as key and the session secret as message, stores the MAC on the session;

7. The session secret is sent as the message content in a push request to the push service provider (Apple or Google). This push send request also contains the application's unique push address as passed in 3 and a package name or certificate tying this to a specific application as published trough the App Store (Google Play or Apple Appstore);

8. The session secret is delivered by the push channel provider to the application (the push channel provider guarantees that only the application with a matching publisher/package name can receive the push message);

9. The application calculates a HMAC using the message key as key and the session secret as message;

10. The application sends HMAC from step 9 to the backend server; and

11. The backend server compares the client calculated HMAC with the server calculated HMAC. If they match the server knows that the client was the recipient of the session secret and that the recipient is the intended application as published in the App Store.

One embodiment of the present invention includes a system for completing a sales transaction, comprising: an application on a mobile device and a backend server; the application generates a message key, encrypts the message key with a public key, and sends the encrypted message key to the backend server; the backend server uses a private key matching the public key to decrypt the encrypted message key and generates a session secret; the backend server calculates a first message authentication code using the decrypted message key and the session secret and sends the first message authentication code and the session secret to a push service provider; the push service provider sends the session secret to the application; the application calculates a second message authentication code using the message key and the session secret and sends the second message authentication code to the backend server; and the backend server compares the second message authentication code with the first message authentication code to determine if they match.

In variations of this embodiment, the application may randomly generate the message key, the application may be provisioned with the public key when the application is published. In the same or alternative embodiments, the application may be provisioned with a push address, the application may send the push address with the encrypted message key to the backend server, and the application may send a certificate tying the push address to a specific application with the encrypted message key to the backend server.

Again in the same or alternative embodiments, the backend server may calculate a message authentication code using the message key as a key and the session secret as a message, the second message authentication code may be a hash message authentication code, and if the second message authentication code matches the first message authentication code the sales transaction is allowed to proceed or, if the second message authentication code matches the first message authentication code, the mobile device is authenticated.

### SafetyNet

SafetyNet Attestation is simply device and app attestation done remotely. It is an anti-abuse API which when added with the abuse detection system of an app checks whether it is running on a genuine Android device. The app security mechanism checks the device's hardware and software environment to create a cryptographically signed attestation.

The API determines the integrity issues on the device and compares the corresponding device profile against the whitelisted device models having Google-approved device profiles. The software backed and hardware-based device profile can be considered compatible only if it matches up with any of the approved profiles in the reference list. A device is considered as approved by Google if it passes the Android Compatibility Test Suite (CTS). Thus, by comparing the device profile against the CTS standards, the API verifies the following:
Whether the device is rooted or not;
Whether the device is monitored;
Whether the bootloader has been unlocked;
Whether the device has recognized hardware parameters;
Whether the software is Android compatible; and
Whether the device is free form malicious apps.

### Context Binding

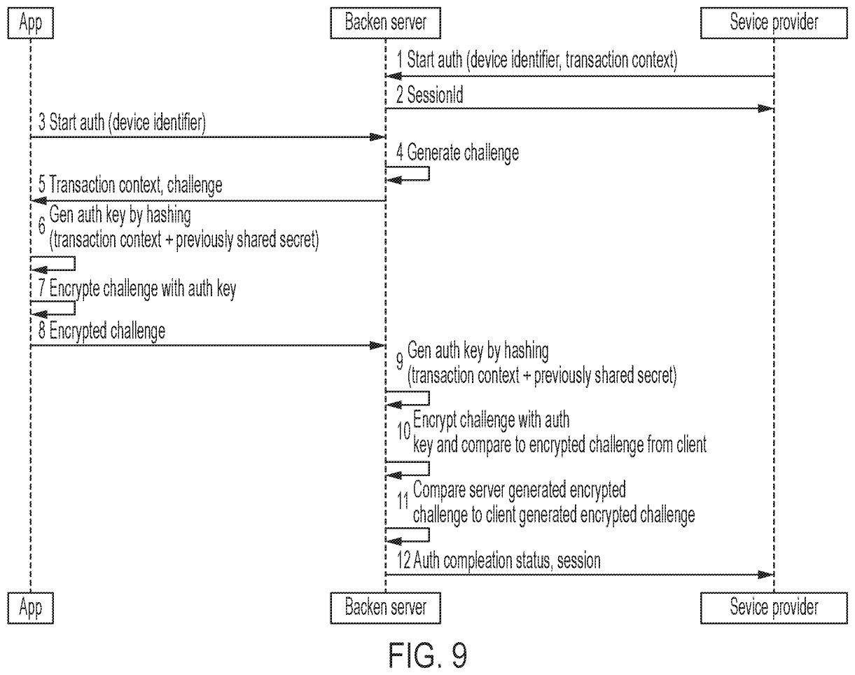

Context binding requires cryptographically tying the context of a transaction to the authentication operation. As shown in FIG. 9, the process proceeds as follows:

1. The service provider starts the authentication process and passes in the transaction context for the authentication;

2. The backend server responds with a session id for this auth session;

3. The client calls the backend server and identifies itself;

4. The backend server generates a random auth challenge;

5. The backend server responds with the transaction context and the auth challenge;

6. The client generates an auth key by hashing a previously shared secret and the transaction context;

7. The client encrypts the challenge from 5 with the auth key from 6;

8. The client passes the encrypted challenge from 7 to the backend server;

9. The backend server generates an auth key by hashing a previously shared secret and the transaction context;

10. The backend server encrypts the auth challenge from 4 with the auth key from 9;

11. The backend server compares the server generated encrypted challenge with the client generated encrypted challenge. If they are equal, the client had both the correct shared secret and transaction context; and

12. The backend server passes the auth completion status to the service provider.

The present invention is directed to improved methods and systems for, among other things, cross channel authentication and/or verification. The configuration and use of the presently preferred embodiments are discussed in detail below. It should be appreciated, however, that the present invention provides many applicable inventive concepts that can be embodied in a wide variety of contexts other than authentication or verification. Accordingly, the specific embodiments discussed are merely illustrative of specific ways to make and use the invention, and do not limit the scope of the invention. In addition, the following terms shall have the associated meaning when used herein:

While the present system and method has been disclosed according to the preferred embodiment of the invention, those of ordinary skill in the art will understand that other embodiments have also been enabled. Even though the foregoing discussion has focused on particular embodiments, it is understood that other configurations are contemplated. In particular, even though the expressions “in one embodiment” or “in another embodiment” are used herein, these phrases are meant to generally reference embodiment possibilities and are not intended to limit the invention to those particular embodiment configurations. These terms may reference the same or different embodiments, and unless indicated otherwise, are combinable into aggregate embodiments. The terms “a”, “an” and “the” mean “one or more” unless expressly specified otherwise. The term “connected” means “communicatively connected” unless otherwise defined.

When a single embodiment is described herein, it will be readily apparent that more than one embodiment may be used in place of a single embodiment. Similarly, where more than one embodiment is described herein, it will be readily apparent that a single embodiment may be substituted for that one device.

In light of the wide variety of authentication and transaction management systems known in the art, the detailed embodiments are intended to be illustrative only and should not be taken as limiting the scope of the invention. Rather, what is claimed as the invention is all such modifications as may come within the spirit and scope of the following claims and equivalents thereto.

None of the description in this specification should be read as implying that any particular element, step or function is an essential element which must be included in the claim scope. The scope of the patented subject matter is defined only by the allowed claims and their equivalents. Unless explicitly recited, other aspects of the present invention as described in this specification do not limit the scope of the claims.

To aid the Patent Office and any readers of any patent issued on this application in interpreting the claims appended hereto, the applicant wishes to note that it does not intend any of the appended claims or claim elements to invoke 35 U.S.C. 112(f) unless the words “means for” or “step for” are explicitly used in the particular claim.

1. A system for completing a sales transaction, comprising:
an application on a mobile device;
a backend server; and
the application generates a message key, encrypts the message key with a public key, and sends the encrypted message key to the backend server;
the backend server uses a private key matching the public key to decrypt the encrypted message key and generates a session secret;
the backend server calculates a first message authentication code using the decrypted message key and the session secret and sends the push address and the session secret to a push service provider;
the push service provider sends the session secret to the application;
the application calculates a second message authentication code using the message key and the session secret and sends the second message authentication code to the backend server; and
the backend server compares the second message authentication code with the first message authentication code to determine if they match.

2. The system for completing a sales transaction of claim 1, wherein the application randomly generates the message key.

3. The system for completing a sales transaction of claim 1, wherein the application is provisioned with the public key when the application was published.

4. The system for completing a sales transaction of claim 1, wherein the application is provisioned with a push address.

5. (canceled)

6. (canceled)

7. The system for completing a sales transaction of claim 1, wherein the backend server calculates a message authentication code using the message key as a key and the session secret as a message.

8. The system for completing a sales transaction of claim 1, wherein the second message authentication code is a hash message authentication code.

9. The system for completing a sales transaction of claim 1, wherein if the second message authentication code matches the first message authentication code the sales transaction is allowed to proceed.

10. The system for completing a sales transaction of claim 1, wherein if the second message authentication code matches the first message authentication code the mobile device is authenticated.
